(19) United States
(12) Patent Application Publication
(10) Pub. No.: US 2026/0007731 A1
(43) Pub. Date: Jan. 8, 2026

(54) INTRANASAL POLYSACCHARIDE CONJUGATE NANOMULSION VACCINES AND METHODS OF USING THE SAME

(71) Applicant: BLUEWillow Biologics, Inc., Ann Arbor, MI (US)

(72) Inventors: Vira BITKO, Ann Arbor, MI (US); Ali FATTOM, Plymouth, MI (US); Shyamala GANESAN, Novi, MI (US)

(73) Assignee: BlueWillow Biologics, Inc., Ann Arbor, MI (US)

(21) Appl. No.: 18/724,898

(22) PCT Filed: Jan. 4, 2023
(86) PCT No.: PCT/US2023/010121

§ 371 Date: Jun. 27, 2024

Related U.S. Application Data
(60) Provisional application No. 63/296,726, filed on Jan. 5, 2022.

(51) Int. Cl. A61K 39/09; A61K 39/00; A61K 39/02; A61K 39/095; A61K 39/112; A61P 31/04

(52) U.S. Cl. CPC A61K 39/092; A61K 39/0216; A61K 39/0275; A61K 39/0283; A61K 39/095; A61P 31/04; A61K 2039/543; A61K 2039/55566; A61K 2039/55583; A61K 2039/575; A61K 2039/6037

(57) ABSTRACT

The present invention relates to intranasal bacterial polysaccharide conjugate nanoemulsion vaccines and methods of using the same for inducing an immune response to a bacterial polysaccharide.

### CROSS-REFERENCE TO RELATED APPLICATIONS

This application is the U.S. National Stage of International Patent Application No. PCT/US2023/010121, filed Jan. 4, 2023, which claims priority from U.S. Provisional Patent Application No. 63/296,726, filed Jan. 5, 2022. The contents of these applications are incorporated herein by reference in their entireties.

### FIELD OF THE APPLICATION

The present application relates to intranasal vaccine compositions comprising a bacterial polysaccharide conjugate and a nanoemulsion adjuvant, and methods of using the same.

### BACKGROUND OF THE INVENTION

Polysaccharide protein conjugate vaccines are primarily used for the prevention of bacterial infections. Some pathogenic bacteria are covered with a polysaccharide capsule that primarily helps protect the bacteria from phagocytosis, or uptake of the bacteria by immune cells. The production of specific antibodies to the polysaccharide coat in an infected person can increase phagocytosis of bacteria, thus stimulating an immune response. Therefore, vaccination using polysaccharides from pathogenic bacteria is a potential strategy for boosting host immunity.

The polysaccharides that cover bacteria vary greatly even within a single species of bacteria. For instance, for Streptococcus pneumoniae, a bacterium that commonly causes pneumonia, there are more than 90 different serotypes due to variation in the bacterial polysaccharide coat. Therefore, polysaccharide vaccines often consist of a panel of polysaccharides to increase protection.

Although polysaccharides are immunogenic on their own, conjugation of polysaccharides to protein carriers has been used to improve immunogenicity. The carrier protein can be either a related protein antigen from the target pathogen, boosting the specific immune response to that pathogen, or a generally immunogenic protein that serves more as an adjuvant or general immune response stimulant.

Prior teachings related to nanoemulsions are described in U.S. Pat. No. 7,314,624, which is directed to methods of inducing an immune response to an immunogen comprising treating a subject via a mucosal surface with a combination of an immunogen and a nanoemulsion. However, none of these references teach the methods, compositions and kits of the present invention.

In particular, U.S. Pat. No. 7,314,624 describes nanoemulsion vaccines. Other nanoemulsion vaccines are described, for example, in U.S. Pat. Nos. 9,144,606 (influenza), U.S. Pat. No. 10,525,121 (influenza), U.S. Pat. No. 9,492,525 (RSV), U.S. Pat. No. 9,561,272 (RSV), U.S. Pat. No. 10,596,251 (RSV), U.S. Pat. No. 10,206,996 (HSV), U.S. Pat. No. 11,147,869 (HSV), U.S. Pat. No. 8,877,208 (multivalent vaccines), U.S. Pat. No. 9,415,006 (multivalent vaccines), U.S. Pat. No. 9,839,685 (HIV), U.S. Pat. No. 10,138,279 (anthrax), U.S. Pat. No. 8,668,911 (streptococcus), and U.S. Pat. No. 11,083,788 (allergic and inflammatory disease). However, these references do not teach the ability to induce a protective immune response to a bacterial pathogen using a combination of a nanoemulsion vaccine adjuvant and a bacterial polysaccharide conjugate.

There remains a need in the art for an effective bacterial vaccines and methods of making and using the same. The present invention satisfies these needs.

### SUMMARY OF THE INVENTION

The present disclosure addresses drawbacks of previously-known approaches by providing intranasal bacterial vaccine compositions and methods of using the same.

An intranasal bacterial vaccine composition may include (a) a polysaccharide-antigen conjugate comprising: (i) one or more polysaccharides from at least one polysaccharide-encapsulated bacteria, and (ii) a carrier protein covalently linked to one or more polysaccharides; and (b) a nanoemulsion adjuvant comprising: (i) droplets having an average diameter of less than about 1000 nm; (ii) an aqueous phase; (iii) about 1% to about 80% of at least one pharmaceutically acceptable oil; (iv) about 0.001% to about 10% of at least one surfactant, wherein the surfactant is a polyoxyethylene nonionic surfactant, a cationic quaternary ammonium compound, or a combination thereof; and (v) about 0.1% to about 50% of at least one organic solvent, wherein the organic solvent is an alcohol.

An intranasal bacterial vaccine composition of the present disclosure may include a panel of at least two polysaccharides from two different bacterial serotypes from the same bacterial genus and species. An intranasal bacterial vaccine composition of the present disclosure may include one or more polysaccharides from bacteria selected from the group consisting of Haemophilus, Neissera, Streptococcus, Shigella, Salmonella, and Porphyromonas. An intranasal bacterial vaccine composition of described herein may include polysaccharides from bacteria selected from the group consisting of Haemophilus influenzae B (Hib), Neissera meningitides, Streptococcus pneumonia (pneumococcus), Shigella dysenteriae, Shigella flexneri, Shigella boydii, Shigella sonnei, Salmonella typhi, Salmonella paratyphi, and Salmonella enterica, and Porphyromonas gingivalis. An intranasal bacterial vaccine composition of the present disclosure may include polysaccharides from at least one polysaccharide-encapsulated bacteria selected from the group consisting of: (a) one or more Streptococcus pneumonia serotypes; (b) a combination of serotypes 1, 2, 3, 4, 5, 6B, 7F, 8, 9N, 9V, 10A, 11A, 12F, 14, 15B, 17F, 18C, 19F, 19A, 20, 22F, 23F, and 33F of Streptococcus pneumonia; (c) a combination of serotypes 1, 3, 4, 5, 6A, 6B, 7F, 9V, 14, 18C, 19A, 19F, and 23F Streptococcus pneumonia; (d) one or more of serotypes 19A, 6, 3, 23F of Streptococcus pneumonia; (e) one or more of serotypes 4, 6B, 9V, 14, 18C, 19F, and 23F of Streptococcus pneumonia; (f) one or more of serotypes 6A, 19A, and 15B/C of Streptococcus pneumonia; and (g) one or more of serotypes 1, 2, 3, 4, 5, 6A, 6B, 6C, 7C, 7F, 8, 9A, 9N, 9A/N, 9N/L, 9V, 10A, 11A, 12F, 14, 15A, 15B, 15A/B, 15C, 16F, 17F, 18B, 18C, 19A, 19F, 19A, 20, 21A, 22F, 23A, 23B, 23 A/B, 23F, 24F, 31, 33F, 35A/B/C, 35B, 38 of Streptococcus pneumonia. An intranasal bacterial vaccine composition of the present disclosure may include polysaccharides from at least one polysaccharide-encapsulated bacteria selected from the group consisting of: (a) one or more serogroups of Neisseria meningitides; (b) one or more of serogroups A, B, C, W, W-135, X, and Y of Neisseria meningitides; and (c) one or more of serogroups A, B, C, D, H, I, K, N, W, W-135, X, and Y of Neisseria meningitides.

An intranasal bacterial vaccine composition of the present disclosure may also include polysaccharides from at least one polysaccharide-encapsulated bacteria selected from the group consisting of: (a) one or more serogroups of Shigella dysenteriae, (b) one or more serogroups of Shigella flexneri; (c) one or more serogroups of Shigella boydii; and/or (d) one or more serogroups of Shigella sonnei. An intranasal bacterial vaccine composition of the present disclosure may include polysaccharides from at least one polysaccharide-encapsulated bacteria selected from the group consisting of: (a) one or more serotypes of Salmonella enterica; (b) Salmonella enterica serotype Enteritidis; (c) Salmonella enterica serotype Typhimurium; (d) one or more serotypes of Salmonella typhi; (e) one or more serotypes of Salmonella typhi; and/or (f) one or more serotypes of Salmonella paratyphi. An intranasal bacterial vaccine composition of the present disclosure may include polysaccharides from at least one polysaccharide-encapsulated bacteria from one or more serotypes of Porphyromonas gingivalis. Further, an intranasal bacterial vaccine composition of the present disclosure may include polysaccharides, wherein the amount of the polysaccharide per dose and per bacterial strain is greater than about 1 μg and less than about 40 μg.

An intranasal bacterial vaccine composition of the present disclosure may include a carrier protein. An intranasal bacterial vaccine composition of the present disclosure may also include a carrier protein selected from the group consisting of diphtheria toxoid (DT), CRM197 diphtheria toxin, tetanus toxoid (TT), CRM197, Haemophilus protein D (PD), and the outer membrane protein complex of serogroup B meningococcus (OMPC).

In addition, an intranasal bacterial vaccine composition of the present disclosure may include a polysaccharide-conjugated antigen comprising bacterial capsular polysaccharides (CPSs).

An intranasal bacterial vaccine composition of the present disclosure may include a nanoemulsion adjuvant. In some embodiments, a nanoemulsion adjuvant and/or a vaccine composition: (a) is not systemically toxic to the subject; and/or (b) produces minimal or no inflammation upon administration. An intranasal bacterial vaccine composition of the present disclosure may include a nanoemulsion adjuvant comprising droplets having an average diameter selected from the group consisting of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, greater than about 125 nm and less than about 600 nm, and any combination thereof.

In addition, an intranasal bacterial vaccine composition of the present disclosure may include a nanoemulsion adjuvant, wherein: (a) the nanoemulsion adjuvant comprises droplets having an average diameter of less than about 800 nm; or (b) the nanoemulsion adjuvant comprises droplets having an average diameter of less than about 600 nm; or (c) the nanoemulsion adjuvant comprises droplets having an average diameter of less than about 500 nm; or (d) the nanoemulsion adjuvant comprises droplets having an average diameter of less than about 400 nm.

In some embodiments, an intranasal bacterial vaccine composition of the present disclosure may include a nanoemulsion adjuvant, wherein (a) the nanoemulsion adjuvant comprises at least one cationic surfactant and at least one polyoxyethylene nonionic surfactant; (b) the nanoemulsion adjuvant comprises at least one cationic surfactant and at least one polyoxyethylene nonionic surfactant which is polysorbate 20, polysorbate 80, poloxamer 188, poloxamer 407, or a combination thereof; (c) the nanoemulsion adjuvant comprises at least one cationic surfactant and at least one polyoxyethylene nonionic surfactant which is polysorbate 20, polysorbate 80, poloxamer 188, poloxamer 407, or a combination thereof, and wherein the polyoxyethylene nonionic surfactant is present at about 0.01% to about 5.0%, or at about 0.1% to about 3%; (d) the nanoemulsion adjuvant comprises at least one cationic surfactant and at least one polyoxyethylene nonionic surfactant, and the non-ionic surfactant is present in a concentration of about 0.05% to about 10%, about 0.05% to about 7.0%, about 0.1% to about 7%, or about 0.5% to about 4%; (e) the nanoemulsion adjuvant comprises at least one cationic surfactant and at least one polyoxyethylene nonionic surfactant, wherein the cationic surfactant is present in a concentration of about 0.05% to about 2% or about 0.01% to about 2%; or (f) any combination of (a)-(f).

Provided herein are vaccine compositions, wherein administration of the vaccine composition to a subject results in seroconversion of the subject after a single administration of the vaccine composition.

In some embodiments, an intranasal bacterial vaccine composition of the present disclosure includes an alcohol, wherein the alcohol is: (a) selected from the group consisting of a C1-C12 alcohol, diol, triol, and combinations thereof; (b) selected from the group consisting of a nonpolar solvent, a polar solvent, a protic solvent, an aprotic solvent, semi-synthetic derivatives thereof, and combinations thereof; (c) selected from the group consisting of ethanol, methanol, isopropyl alcohol, glycerol, n-butanol, butylene glycol, perfumers alcohols, isopropanol, n-propanol, propylene glycols, glycerol, sorbitol, and any combination thereof; or (d) any combination of (a), (b), and/or (c).

In some embodiments, an intranasal bacterial vaccine composition of the present disclosure includes an oil, wherein the oil: (a) is selected from the group consisting of animal oil, vegetable oil, natural oil, synthetic oil, hydrocarbon oils, silicone oils, and semi-synthetic derivatives there; or (b) is selected from the group consisting of soybean, avocado, squalene, olive, canola, corn, rapeseed, safflower, sunflower, fish, flavor, and water insoluble vitamins.

In some embodiments, an intranasal bacterial vaccine composition of the present disclosure includes a nonionic polyoxyethylene surfactant selected from the group consisting of polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, semi-synthetic derivatives thereof, and mixtures thereof.

In some embodiments, an intranasal bacterial vaccine composition of the present disclosure includes a cationic quaternary ammonium compound selected from the group consisting of an alkyl trimethyl ammonium chloride compound, a dialkyl dimethyl ammonium chloride compound, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Cetylpyridinium chloride, Dimethyldioctadecylammonium bromide, Dodecylethyldimethylammonium bromide, Dodecyltrimethylammonium bromide, Ethylhexadecyldimethylammonium bromide, Girard's reagent T, Hexadecyltrimethylammonium bromide, N,N′, N′-Polyoxyethylene (10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C.sub. 12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof.

An intranasal bacterial vaccine composition of the present disclosure may include chitosan. An intranasal bacterial vaccine composition of the present disclosure may include glucan. In some embodiments, an intranasal bacterial vaccine composition of the present disclosure includes an aqueous phase comprising Phosphate Buffered Saline (PBS).

An intranasal bacterial vaccine composition of the present disclosure may be formulated: (a) into a dosage form selected from the group consisting of a liquid dispersion, aerosol, and suspensions; and/or (b) into a controlled release formulation, sustained release formulation, or immediate release formulation.

Provided herein is a method for inducing an enhanced immunity against disease caused by a polysaccharide-encapsulated bacteria comprising the step of administering to a subject an effective amount of the intranasal bacterial vaccine composition of any one of claims 1-24. In some embodiments, a method for inducing an enhanced immunity against disease caused by a polysaccharide-encapsulated bacteria involves the subject producing a protective immune response after at least a single administration of the vaccine composition. Such an immune response may be protective against one or more serotypes of the bacteria. In some such embodiments, the subject may exhibit a higher titer of bacteria-specific antibodies relative to a subject not administered the intranasal bacterial vaccine composition. In some embodiments, the bacteria-specific antibodies may include IgG antibodies. In some embodiments, the bacteria-specific antibodies may include IgA antibodies.

In some embodiments, a subject exposed to an intranasal bacterial vaccine composition of the present disclosure may exhibit elevated serum levels of IFN-γ as compared to the levels found within a subject not administered the intranasal bacterial vaccine composition. In some embodiments, a subject exposed to an intranasal bacterial vaccine composition of the present disclosure may exhibit elevated serum levels of TNF-α as compared to the levels found within a subject not administered the intranasal bacterial vaccine composition. In some embodiments, a subject exposed to an intranasal bacterial vaccine composition of the present disclosure may exhibit elevated serum levels of IL-4 as compared to the levels found within a subject not administered the intranasal bacterial vaccine composition. In some embodiments, a subject exposed to an intranasal bacterial vaccine composition of the present disclosure may exhibit elevated serum levels of IL-5 as compared to the levels found within a subject not administered the intranasal bacterial vaccine composition. In some embodiments, a subject exposed to an intranasal bacterial vaccine composition of the present disclosure may exhibit elevated serum levels of IL-17 as compared to the levels found within a subject not administered the intranasal bacterial vaccine composition. In some embodiments, the elevated levels are measured in the lung and/or spleen of the subject.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### DESCRIPTION OF THE FIGURES

FIG. 1: Shows the study design for Example 1, in which CD-1 mice (n=7 per group, with two groups) were inoculated intranasally at days 1, 21, and 42 with 4 μg/dose/animal with a NE01 nanoemulsion vaccine composition comprising Pneum conjugate (Pneum Conj/NE01). The 4 μg dose is based on the quantity of polysaccharide. Animals were sacrificed at day 70.

FIGS. 2A-D: Show Pn18c-specific IgG and IgA titers in serum and BAL fluid samples from Pneum Conj/NE01-immunized and control (PBS-administered) mice, as measured by ELISA. FIG. 2A shows the ELISA results for Pn18c-specific serum IgG, with the endpoint titer (EPT) shown for the immunized group and the PBS (control) group. FIG. 2B shows the ELISA results for Pn18c-specific serum IgA, with EPT shown for the immunized group and the control group. FIG. 2C shows the ELISA results for Pn18c-specific BAL IgG, with the OD450 at 1:5 dilution shown for the vaccine group and the control group. Finally, FIG. 2D shows the ELISA results for Pn18c-specific BAL IgA, with the OD450 at 1:5 dilution shown for the vaccine group and the PBS group.

FIGS. 3A-D: Show CRM-specific IgG and IgA titers in serum and BAL fluid samples from Pneum Conj/NE01-immunized and control (PBS-administered) mice, as measured by ELISA. FIG. 3A shows the ELISA results for CRM-specific serum IgG, with the endpoint titer (EPT) shown for the immunized group and the PBS (control) group. FIG. 3B shows the ELISA results for CRM-specific serum IgA, with EPT shown for the immunized group and the control group. FIG. 3C shows the ELISA results for CRM-specific BAL IgG, with the OD450 at 1:5 dilution shown for the vaccine group and the control group. Finally, FIG. 3D shows the ELISA results for CRM-specific BAL IgA, with the OD450 at 1:5 dilution shown for the vaccine group and the PBS group.

FIGS. 4A and B show the results of evaluating lung tissue for the Th1 cytokines IFNγ and TNFα via Luminex assays. FIG. 4A shows IFNγ levels in single cell suspensions of lung tissue from Pneum conj/NE01-immunized mice and control mice following stimulation of the cells by CRM or Pn18c. FIG. 4B shows the same for TNFα levels.

FIGS. 5A and B show the results of evaluating lung tissue for the Th2 cytokines IL4 and IL5 via Luminex assays. FIG. 5A shows IL4 levels in single cell suspensions of lung tissue from Pneum conj/NE01-immunized mice and control mice following stimulation of the cells by CRM or Pn18c. FIG. 5B shows the same for IL5 levels.

FIG. 6 shows the results of evaluating lung tissue for the Th17 cytokine IL17 via Luminex assay. Shown are IL17 levels in single cell suspensions of lung tissue from Pneum conj/NE01-immunized mice and control mice following stimulation of the cells by CRM or Pn18c.

FIGS. 7A and B show the results of evaluating spleen tissue for the Th1 cytokines IFNγ and TNFα via Luminex assays. FIG. 7A shows IFNγ levels in single cell suspensions of spleen tissue from Pneum conj/NE01-immunized mice and control mice following stimulation of the cells by CRM or Pn18c. FIG. 7B shows the same for TNFα levels.

FIGS. 8A and B show the results of evaluating spleen tissue for the Th2 cytokines IL4 and IL5 via Luminex assays. FIG. 8A shows IL4 levels in single cell suspensions of spleen tissue from Pneum conj/NE01-immunized mice and control mice following stimulation of the cells by CRM or Pn18c. FIG. 8B shows the same for IL5 levels.

FIG. 9 shows the results of evaluating spleen tissue for the Th17 cytokine IL17 via Luminex assay. Shown are IL17 levels in single cell suspensions of spleen tissue from Pneum conj/NE01-immunized mice and control mice following stimulation of the cells by CRM or Pn18c.

FIGS. 10A-D: Show the results of evaluating homing of IgG- and IgA-producing B cells to lungs and spleens using an ELISpot Assay. FIG. 10A shows a quantification of the levels of IgG-producing B cells in single cell suspensions of lung tissue samples from Pneum conj/NE01-immunized mice and PBS-administered (naïve) mice, measured in spot forming units (SFU)/million cells. FIG. 10B shows a quantification of the levels of IgA-producing B cells in single cell suspensions of lung tissue samples from Pneum conj/NE01-immunized mice and PBS-administered (naïve) mice. FIG. 10C shows a quantification of the levels of IgG-producing B cells in single cell suspensions of spleen tissue samples from Pneum conj/NE01-immunized mice and PBS-administered (naïve) mice. FIG. 10D shows a quantification of the levels of IgA-producing B cells in single cell suspensions of spleen tissue samples from Pneum conj/NE01-immunized mice and PBS-administered (naïve) mice.

FIGS. 11A and B show the results of an OREP18C OPKA assay. FIG. 11A shows the Total CFU for each of the vaccine and PBS (control) groups. FIG. 11B shows the OPKA OREP18C (Pn18C) results for each of the vaccine and control groups.

### DETAILED DESCRIPTION OF THE INVENTION

### I. Overview

The present invention provides methods, compositions and kits for the stimulation of an immune response to a bacterial polysaccharide immunogen. For example, the present disclosure provides vaccine compositions useful for inducing an immune response to a bacterial polysaccharide immunogen, such as, for example, a bacterial polysaccharide of Streptococcus pneumoniae, and methods and kits for the same. A vaccine composition of the present invention may include a bacterial polysaccharide conjugated to a protein carrier. For example, in some embodiments, a vaccine composition of the invention may comprise a Pn18c polysaccharide from Streptococcus pneumoniae conjugated to the carrier protein CRM197, which is a non-toxic mutant of diphtheria toxin. Such a so-called conjugate vaccine may have advantages relative to traditional polysaccharide vaccines, including but not limited to enhanced immunogenicity. In some embodiments of the present disclosure, a vaccine composition may be formulated in a nanoemulsion adjuvant. A nanoemulsion polysaccharide conjugate vaccine of the present invention maybe, in some embodiments, be delivered to a subject intranasally (IN).

Most significantly, as detailed in Example 1, the present inventors showed that nanoemulsion polysaccharide conjugate vaccines are capable of eliciting a protective immune response in mice following IN administration. Such an immune response included stimulation of immunoglobulin and cytokine production in cells of the lungs and spleen, and homing of immunoglobulin-producing B cells to the lungs and spleens of immunized mice. Further, it was shown that sera of mice immunized with a nanoemulsion polysaccharide conjugate S. pneumoniae vaccine harbored potent S. pneumoniae killing activity, indicating that the vaccine may be highly protective against infection by S. pneumoniae.

The present inventors surprisingly discovered that polysaccharide conjugated vaccines can be administered intranasally if formulated in a nanoemulsion adjuvant. Moreover, such formulations were found to elicit a protective immune response.

Exemplary methods of conjugation that can be used to make the polysaccharide conjugated vaccines described herein, include, but are not limited to, those for Pn18c conjugation to CRM197. Protein conjugation may be performed using any conventional protein conjugation technique. Methods of protein conjugation are well-known in the art. Exemplary methods of protein conjugation include, but are not limited to, those described in Sarkar & Jayaraman, “Glycoconjugations of Biomolecules by Chemical Methods,” Front. Chem., 2020 (doi.org/10.3389/fchem.2020.570185); Berti & Adamo, “Antimicrobial glycoconjugate vaccines: an overview of classic and modern approaches for protein modification,” Chem. Soc. Rev., 47:9015 (2018); Ada & Isaacs, “Carbohydrate-protein conjugate vaccines,” Clin. Microbiol. Infect., 9(2):79-85 (2003); Lees et al., “Versatile and efficient synthesis of protein-polysaccharide conjugate vaccines using aminooxy reagents and oxime chemistry,” Vaccine, 24(6):716-29 (2006), each of which is incorporated by reference in its entirety.

The methods comprise intranasally administering to a subject a nanoemulsion bacterial polysaccharide conjugate vaccine comprising a combination of a bacterial polysaccharide conjugate and a nanoemulsion vaccine adjuvant. The nanoemulsion vaccine adjuvant comprises droplets having an average diameter of less than about 1000 nm. The nanoemulsion vaccine adjuvant further comprises (a) an aqueous phase, (b) at least one oil, (c) at least one surfactant, (d) at least one organic solvent, and (e) optionally comprising at least one chelating agent, or any combination thereof. In another embodiment of the invention, the nanoemulsion vaccine adjuvant lacks an organic solvent.

In one embodiment, the subject is selected from adults, elderly subjects, juvenile subjects, infants, high risk subjects, pregnant women, and immunocompromised subjects.

The nanoemulsion vaccine adjuvant serves to: (1) bring the antigen—the substance that stimulates the specific protective immune response—into contact with the immune system and influence the type of immunity produced, as well as the quality of the immune response (magnitude or duration); (2) decrease the toxicity of certain antigens; (3) reduce the amount of antigen needed for a protective response; (4) reduce the number of doses required for protection; (5) enhance immunity in poorly responding subsets of the population and/or (7) provide solubility to some vaccines components. The nanoemulsion vaccine adjuvants are particularly useful for adjuvanting bacterial polysaccharide conjugate vaccines.

Nanoemulsions are oil-in-water emulsions composed of nanometer sized droplets with surfactant(s) at the oil-water interface. Because of their size, the nanoemulsion droplets are pinocytosed by dendritic cells triggering cell maturation and efficient antigen presentation to the immune system. When mixed with different antigens, nanoemulsion adjuvants elicit and up-modulate strong humoral and cellular T_{H}1-type responses as well as mucosal immunity (Makidon et al., “Pre-Clinical Evaluation of a Novel Nanoemulsion-Based Hepatitis B Mucosal Vaccine,” PLoS ONE. 3(8): 2954; 1-15 (2008); Hamouda et al., “A Novel Nanoemulsion Adjuvant Enhancing The Immune Response from Intranasal Influenza Vaccine in Mice in National Foundation for Infectious Disease,” 11th Annual Conference on Vaccine Research. Baltimore, MD (2008); Myc et al., “Development of immune response that protects mice from viral pneumonitis after a single intranasal immunization with influenza A virus and nanoemulsion,” Vaccine, 21(25-26):3801-14 (2003); Bielinska et al., “Mucosal Immunization with a Novel Nanoemulsion-Based Recombinant Anthrax Protective Antigen Vaccine Protects against Bacillus anthracis Spore Challenge,” Infect Immun., 75(8): 4020-9 (2007); Bielinska et al., “Nasal Immunization with a Recombinant HIV gp120 and Nanoemulsion Adjuvant Produces Th1 Polarized Responses and Neutralizing Antibodies to Primary HIV Type 1 Isolates,” AIDS Research and Human Retroviruses, 24(2): 271-81 (2008); Bielinska et al., “A Novel, Killed-Virus Nasal Vaccinia Virus Vaccine,” Clin. Vaccine Immunol., 15(2): 348-58 (2008)).

The nanoemulsion vaccine can be formulated into any pharmaceutically acceptable dosage form which can be administered intranasally, such as a liquid dispersion, aerosol, or a suspension. Further, the nanoemulsion vaccine may be a controlled release formulation, sustained release formulation, immediate release formulation, or any combination thereof.

In one embodiment of the invention, the nanoemulsion vaccine adjuvant comprises droplets having an average diameter of less than about 1000 nm and: (a) an aqueous phase; (b) about 1% oil to about 80% oil; (c) about 0.1% to about 50% organic solvent; (d) about 0.001% to about 10% of a surfactant or detergent; or (e) any combination thereof. In another embodiment of the invention, the nanoemulsion vaccine adjuvant comprises: (a) an aqueous phase; (b) about 1% oil to about 80% oil; (c) about 0.1% to about 50% organic solvent; (d) about 0.001% to about 10% of a surfactant or detergent. In another embodiment of the invention, the nanoemulsion vaccine adjuvant lacks an organic solvent.

In one embodiment, the nanoemulsion vaccine adjuvant comprises a cationic surfactant which is cetylpyridinium chloride (CPC). CPC may have a concentration in the nanoemulsion vaccine of less than about 5.0% and greater than about 0.001%, or further, may have a concentration of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, and greater than about 0.010%.

In a further embodiment, the nanoemulsion vaccine adjuvant comprises a non-ionic surfactant, such as a polysorbate surfactant, which may be polysorbate 80 or polysorbate 20, and may have a concentration of about 0.01% to about 5.0%, or about 0.1% to about 3% of polysorbate 80. The intranasal bacterial polysaccharide conjugate nanoemulsion vaccine may further comprise at least one preservative. In another embodiment of the invention, the intranasal bacterial polysaccharide conjugate nanoemulsion vaccine comprises a chelating agent.

In yet another embodiment, the nanoemulsion vaccine adjuvant further comprises an immune modulator, such as chitosan or glucan. An immune modulator can be present in the vaccine composition at any pharmaceutically acceptable amount including, but not limited to, from about 0.001% up to about 10%, and any amount in between, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%.

The immune response of the subject can be measured by determining the titer and/or presence of antibodies against the bacterial polysaccharide conjugate after administration of the intranasal bacterial polysaccharide conjugate nanoemulsion vaccine to evaluate the humoral response to the immunogen. Seroconversion refers to the development of specific antibodies to an immunogen and may be used to evaluate the presence of a protective immune response. Such antibody-based detection is often measured using Western blotting or enzyme-linked immunosorbent (ELISA) assays or hemagglutination inhibition assays (HAI). Persons of skill in the art would readily select and use appropriate detection methods.

Another method for determining the subject's immune response is to determine the cellular immune response, such as through immunogen-specific cell responses, such as cytotoxic T lymphocytes, or immunogen-specific lymphocyte proliferation assay. Additionally, challenge by the pathogen may be used to determine the immune response, either in the subject, or, more likely, in an animal model. A person of skill in the art would be well versed in the methods of determining the immune response of a subject and the invention is not limited to any particular method.

### II. Stability of the Intranasal Bacterial Polysaccharide Conjugate Nanoemulsion Vaccines of the Invention

The intranasal bacterial polysaccharide conjugate nanoemulsion vaccines of the invention can be stable at about 40° C. and about 75% relative humidity for a time period of at least up to about 2 days, at least up to about 2 weeks, at least up to about 1 month, at least up to about 3 months, at least up to about 6 months, at least up to about 12 months, at least up to about 18 months, at least up to about 2 years, at least up to about 2.5 years, or at least up to about 3 years.

In another embodiment of the invention, the intranasal bacterial polysaccharide conjugate nanoemulsion vaccines of the invention can be stable at about 25° C. and about 60% relative humidity for a time period of at least up least up to about 2 days, at least up to about 2 weeks, to about 1 month, at least up to about 3 months, at least up to about 6 months, at least up to about 12 months, at least up to about 18 months, at least up to about 2 years, at least up to about 2.5 years, or at least up to about 3 years, at least up to about 3.5 years, at least up to about 4 years, at least up to about 4.5 years, or at least up to about 5 years.

Further, the intranasal bacterial polysaccharide conjugate nanoemulsion vaccines of the invention can be stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

The intranasal bacterial polysaccharide conjugate nanoemulsion vaccines of the invention can be stable at about −20° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

### III. Intranasal Bacterial Polysaccharide Conjugate Nanoemulsion Vaccines

### A. Droplet Size

The intranasal bacterial polysaccharide conjugate nanoemulsion vaccines of the present invention comprise droplets having an average diameter size of less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 600 nm. In a different embodiment, the droplets have an average diameter size greater than about 50 nm or greater than about 70 nm, and less than or equal to about 125 nm.

### B. Aqueous Phase

The aqueous phase can comprise any type of aqueous phase including, but not limited to, water (e.g., H₂O, distilled water, purified water, water for injection, de-ionized water, tap water) and solutions (e.g., phosphate buffered saline (PBS) solution). In certain embodiments, the aqueous phase comprises water at a pH of about 4 to 10, preferably about 6 to 8. The water can be deionized (hereinafter “DiH₂O”). In some embodiments the aqueous phase comprises phosphate buffered saline (PBS). The aqueous phase may further be sterile and pyrogen free.

### C. Organic Solvents

Organic solvents in the intranasal bacterial polysaccharide conjugate nanoemulsion vaccines of the invention include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the intranasal bacterial polysaccharide conjugate nanoemulsion vaccines include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

### D. Oil Phase

The oil in the intranasal bacterial polysaccharide conjugate nanoemulsion vaccines of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component is different than the oil in the oil phase.

### E. Surfactants

The surfactant in the intranasal bacterial polysaccharide conjugate nanoemulsion vaccines of the invention can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag Gmbh & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thiglycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂ CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-100, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′, N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride (100% C₁₂), Alkyl dimethyl 3,4-dichlorobenzyl ammonium chloride (50% C₁₄, 40% C₁₂, 10% C₁₆), Alkyl dimethyl 3,4-dichlorobenzyl ammonium chloride (55% C₁₄, 23% C₁₂, 20% C₁₆), Alkyl dimethyl benzyl ammonium chloride, Alkyl dimethyl benzyl ammonium chloride (100% C₁₄), Alkyl dimethyl benzyl ammonium chloride (100% C₁₆), Alkyl dimethyl benzyl ammonium chloride (41% C₁₄, 28% C₁₂), Alkyl dimethyl benzyl ammonium chloride (47% C₁₂, 18% C₁₄), Alkyl dimethyl benzyl ammonium chloride (55% C₁₆, 20% C₁₄), Alkyl dimethyl benzyl ammonium chloride (58% C₁₄, 28% C₁₆), Alkyl dimethyl benzyl ammonium chloride (60% C₁₄, 25% C₁₂), Alkyl dimethyl benzyl ammonium chloride (61% C₁₁, 23% C₁₄), Alkyl dimethyl benzyl ammonium chloride (61% C₁₂, 23% C₁₄), Alkyl dimethyl benzyl ammonium chloride (65% C₁₂, 25% C₁₄), Alkyl dimethyl benzyl ammonium chloride (67% C₁₂, 24% C₁₄), Alkyl dimethyl benzyl ammonium chloride (67% C₁₂, 25% C₁₄), Alkyl dimethyl benzyl ammonium chloride (90% C₁₄, 5% C₁₂), Alkyl dimethyl benzyl ammonium chloride (93% C₁₄, 4% C₁₂), Alkyl dimethyl benzyl ammonium chloride (95% C₁₆, 5% C₁₈), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C₁₂₋₁₆), Alkyl dimethyl benzyl ammonium chloride (C₁₂₋₁₈), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C₁₄, 5% C₁₆, 5% C₁₂), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C₁₄), Alkyl dimethyl isopropylbenzyl ammonium chloride (50% C₁₂, 30% C₁₄, 17% C₁₆, 3% C₁₈), Alkyl trimethyl ammonium chloride (58% C₁₈, 40% C₁₆, 1% C₁₄, 1% C₁₂), Alkyl trimethyl ammonium chloride (90% C₁₈, 10% C₁₆), Alkyldimethyl(ethylbenzyl) ammonium chloride (C₁₂₋₁₈), Di-(C₈₋₁₀)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with an particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the intranasal bacterial polysaccharide conjugate nanoemulsion vaccine comprises a cationic surfactant, which can be cetylpyridinium chloride. In other embodiments of the invention, the intranasal bacterial polysaccharide conjugate nanoemulsion vaccine comprises a cationic surfactant, and the concentration of the cationic surfactant is less than about 5.0% and greater than about 0.001%. In yet another embodiment of the invention, the intranasal bacterial polysaccharide conjugate nanoemulsion vaccine comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion vaccine is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion vaccine is less than about 5.0% and greater than about 0.001%.

In another embodiment of the invention, the nanoemulsion vaccine comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.01% to about 5.0%, or the non-ionic surfactant is present in a concentration of about 0.1% to about 3%. In yet another embodiment of the invention, the nanoemulsion vaccine comprises a cationic surfactant present in a concentration of about 0.01% to about 2%, in combination with a nonionic surfactant.

### F. Additional Ingredients

Additional compounds suitable for use in the intranasal bacterial polysaccharide conjugate nanoemulsion vaccines of the invention include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion vaccine, or the additional compounds can be added to the original mixture to be emulsified. In certain of these embodiments, one or more additional compounds are admixed into an existing nanoemulsion composition immediately prior to its use.

Suitable preservatives in the intranasal bacterial polysaccharide conjugate nanoemulsion vaccines of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The intranasal bacterial polysaccharide conjugate nanoemulsion vaccine may further comprise at least one pH adjuster. Suitable pH adjusters in the nanoemulsion vaccine of the invention include, but are not limited to, diethyanolamine, lactic acid, monoethanolamine, triethylanolamine, sodium hydroxide, sodium phosphate, semi-synthetic derivatives thereof, and combinations thereof.

In addition, the intranasal bacterial polysaccharide conjugate nanoemulsion vaccine can comprise a chelating agent. In one embodiment of the invention, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The intranasal bacterial polysaccharide conjugate nanoemulsion vaccine can comprise a buffering agent, such as a pharmaceutically acceptable buffering agent. Examples of buffering agents include, but are not limited to, 2-Amino-2-methyl-1,3-propanediol, ≥99.5% (NT), 2-Amino-2-methyl-1-propanol, ≥99.0% (GC), L-(+)-Tartaric acid, ≥99.5% (T), ACES, ≥99.5% (T), ADA, ≥99.0% (T), Acetic acid, ≥99.5% (GC/T), Acetic acid, for luminescence, ≥99.5% (GC/T), Ammonium acetate solution, for molecular biology, ˜5 M in H2O, Ammonium acetate, for luminescence, ≥99.0% (calc. on dry substance, T), Ammonium bicarbonate, ≥99.5% (T), Ammonium citrate dibasic, ≥99.0% (T), Ammonium formate solution, 10 M in H₂O, Ammonium formate, ≥99.0% (calc. based on dry substance, NT), Ammonium oxalate monohydrate, ≥99.5% (RT), Ammonium phosphate dibasic solution, 2.5 M in H₂O, Ammonium phosphate dibasic, ≥99.0% (T), Ammonium phosphate monobasic solution, 2.5 M in H₂O, Ammonium phosphate monobasic, ≥99.5% (T), Ammonium sodium phosphate dibasic tetrahydrate, ≥99.5% (NT), Ammonium sulfate solution, for molecular biology, 3.2 M in H₂O, Ammonium tartrate dibasic solution, 2 M in H₂O (colorless solution at 20° C.), Ammonium tartrate dibasic, ≥99.5% (T), BES buffered saline, for molecular biology, 2× concentrate, BES, ≥99.5% (T), BES, for molecular biology, ≥99.5% (T), BICINE buffer Solution, for molecular biology, 1 M in H₂O, BICINE, ≥99.5% (T), BIS-TRIS, ≥99.0% (NT), Bicarbonate buffer solution, >0.1 M Na₂CO₃, >0.2 M NaHCO₃, Boric acid, ≥99.5% (T), Boric acid, for molecular biology, ≥99.5% (T), CAPS, ≥99.0% (TLC), CHES, ≥99.5% (T), Calcium acetate hydrate, ≥99.0% (calc. on dried material, KT), Calcium carbonate, precipitated, ≥99.0% (KT), Calcium citrate tribasic tetrahydrate, ≥98.0% (calc. on dry substance, KT), Citrate Concentrated Solution, for molecular biology, 1 M in H₂O, Citric acid, anhydrous, ≥99.5% (T), Citric acid, for luminescence, anhydrous, ≥99.5% (T), Diethanolamine, ≥99.5% (GC), EPPS, ≥99.0% (T), Ethylenediaminetetraacetic acid disodium salt dihydrate, for molecular biology, ≥99.0% (T), Formic acid solution, 1.0 M in H₂O, Gly-Gly-Gly, ≥99.0% (NT), Gly-Gly, ≥99.5% (NT), Glycine, ≥99.0% (NT), Glycine, for luminescence, ≥99.0% (NT), Glycine, for molecular biology, ≥99.0% (NT), HEPES buffered saline, for molecular biology, 2× concentrate, HEPES, ≥99.5% (T), HEPES, for molecular biology, ≥99.5% (T), Imidazole buffer Solution, 1 M in H₂O, Imidazole, ≥99.5% (GC), Imidazole, for luminescence, ≥99.5% (GC), Imidazole, for molecular biology, ≥99.5% (GC), Lipoprotein Refolding Buffer, Lithium acetate dihydrate, ≥99.0% (NT), Lithium citrate tribasic tetrahydrate, ≥99.5% (NT), MES hydrate, ≥99.5% (T), MES monohydrate, for luminescence, ≥99.5% (T), MES solution, for molecular biology, 0.5 M in H₂O, MOPS, ≥99.5% (T), MOPS, for luminescence, ≥99.5% (T), MOPS, for molecular biology, ≥99.5% (T), Magnesium acetate solution, for molecular biology, ˜1 M in H₂O, Magnesium acetate tetrahydrate, ≥99.0% (KT), Magnesium citrate tribasic nonahydrate, ≥98.0% (calc. based on dry substance, KT), Magnesium formate solution, 0.5 M in H₂O, Magnesium phosphate dibasic trihydrate, ≥98.0% (KT), Neutralization solution for the in-situ hybridization for in-situ hybridization, for molecular biology, Oxalic acid dihydrate, ≥99.5% (RT), PIPES, ≥99.5% (T), PIPES, for molecular biology, ≥99.5% (T), Phosphate buffered saline, solution (autoclaved), Phosphate buffered saline, washing buffer for peroxidase conjugates in Western Blotting, 10× concentrate, Piperazine, anhydrous, ≥99.0% (T), Potassium D-tartrate monobasic, ≥99.0% (T), Potassium acetate solution, for molecular biology, Potassium acetate solution, for molecular biology, 5 M in H₂O, Potassium acetate solution, for molecular biology, ˜1 M in H₂O, Potassium acetate, ≥99.0% (NT), Potassium acetate, for luminescence, ≥99.0% (NT), Potassium acetate, for molecular biology, ≥99.0% (NT), Potassium bicarbonate, ≥99.5% (T), Potassium carbonate, anhydrous, ≥99.0% (T), Potassium chloride, ≥99.5% (AT), Potassium citrate monobasic, ≥99.0% (dried material, NT), Potassium citrate tribasic solution, 1 M in H₂O, Potassium formate solution, 14 M in H₂O, Potassium formate, ≥99.5% (NT), Potassium oxalate monohydrate, ≥99.0% (RT), Potassium phosphate dibasic, anhydrous, ≥99.0% (T), Potassium phosphate dibasic, for luminescence, anhydrous, ≥99.0% (T), Potassium phosphate dibasic, for molecular biology, anhydrous, ≥99.0% (T), Potassium phosphate monobasic, anhydrous, ≥99.5% (T), Potassium phosphate monobasic, for molecular biology, anhydrous, ≥99.5% (T), Potassium phosphate tribasic monohydrate, ≥95% (T), Potassium phthalate monobasic, ≥99.5% (T), Potassium sodium tartrate solution, 1.5 M in H2O, Potassium sodium tartrate tetrahydrate, ≥99.5% (NT), Potassium tetraborate tetrahydrate, ≥99.0% (T), Potassium tetraoxalate dihydrate, ≥99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≥99.5% (NT), Sodium acetate solution, for molecular biology, ˜3 M in H₂O, Sodium acetate trihydrate, ≥99.5% (NT), Sodium acetate, anhydrous, ≥99.0% (NT), Sodium acetate, for luminescence, anhydrous, ≥99.0% (NT), Sodium acetate, for molecular biology, anhydrous, ≥99.0% (NT), Sodium bicarbonate, ≥99.5% (T), Sodium bitartrate monohydrate, ≥99.0% (T), Sodium carbonate decahydrate, ≥99.5% (T), Sodium carbonate, anhydrous, ≥99.5% (calc. on dry substance, T), Sodium citrate monobasic, anhydrous, ≥99.5% (T), Sodium citrate tribasic dihydrate, ≥99.0% (NT), Sodium citrate tribasic dihydrate, for luminescence, ≥99.0% (NT), Sodium citrate tribasic dihydrate, for molecular biology, ≥99.5% (NT), Sodium formate solution, 8 M in H₂O, Sodium oxalate, ≥99.5% (RT), Sodium phosphate dibasic dihydrate, ≥99.0% (T), Sodium phosphate dibasic dihydrate, for luminescence, ≥99.0% (T), Sodium phosphate dibasic dihydrate, for molecular biology, ≥99.0% (T), Sodium phosphate dibasic dodecahydrate, ≥99.0% (T), Sodium phosphate dibasic solution, 0.5 M in H₂O, Sodium phosphate dibasic, anhydrous, ≥99.5% (T), Sodium phosphate dibasic for molecular biology, ≥99.5% (T), Sodium phosphate monobasic dihydrate, ≥99.0% (T), Sodium phosphate monobasic dihydrate, for molecular biology, ≥99.0% (T), Sodium phosphate monobasic monohydrate, for molecular biology, ≥99.5% (T), Sodium phosphate monobasic solution, 5 M in H₂O, Sodium pyrophosphate dibasic, ≥99.0% (T), Sodium pyrophosphate tetrabasic decahydrate, ≥99.5% (T), Sodium tartrate dibasic dihydrate, ≥99.0% (NT), Sodium tartrate dibasic solution, 1.5 M in H₂O (colorless solution at 20° C.), Sodium tetraborate decahydrate, ≥99.5% (T), TAPS, ≥99.5% (T), TES, ≥99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate-EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≥99.5% (NT), Triethanolamine, ≥99.5% (GC), Triethylamine, ≥99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≥99.0% (NT), Trizma® base, ≥99.8% (T), Trizma® base, ≥99.8% (T), Trizma® base, for luminescence, ≥99.8% (T), Trizma® base, for molecular biology, ≥99.8% (T), Trizma® carbonate, ≥98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≥99.0% (AT), Trizma® hydrochloride, for luminescence, ≥99.0% (AT), Trizma® hydrochloride, for molecular biology, ≥99.0% (AT), and Trizma® maleate, ≥99.5% (NT).

The intranasal bacterial polysaccharide conjugate nanoemulsion vaccine can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsion vaccines that may readily be diluted with water or another aqueous phase to a desired concentration without impairing their desired properties.

### G. Immune Modulators

As noted above, the intranasal bacterial polysaccharide conjugate nanoemulsion vaccines can further comprise one or more immune modulators. Examples of immune modulators include, but are not limited to, chitosan, glucan, enterotoxin, nucleic acid (CpG motifs), MF59, alum, ASO system, etc. It is within the purview of one of ordinary skill in the art to employ suitable immune modulators in the context of the present invention.

An immune modulator can be present in the vaccine composition at any pharmaceutically acceptable amount including, but not limited to, from about 0.001% up to about 10%, and any amount inbetween, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%.

### IV. Pharmaceutical Compositions

The intranasal bacterial polysaccharide conjugate nanoemulsion vaccines of the invention may be formulated into pharmaceutical compositions that comprise the intranasal bacterial polysaccharide conjugate nanoemulsion vaccine in a therapeutically effective amount and suitable, pharmaceutically-acceptable excipients for pharmaceutically acceptable delivery. Such excipients are well known in the art.

By the phrase “therapeutically effective amount” it is meant any amount of the intranasal bacterial polysaccharide conjugate nanoemulsion vaccines that is effective in preventing, treating or ameliorating a disease caused by the bacterial pathogen associated with the immunogen administered in the composition comprising the intranasal bacterial polysaccharide conjugate nanoemulsion vaccine. By “protective immune response” it is meant that the immune response is associated with prevention, treating, or amelioration of a disease. Complete prevention is not required, though is encompassed by the present invention. The immune response can be evaluated using the methods discussed herein or by any method known by a person of skill in the art.

Intranasal administration includes administration via the nose, either with or without concomitant inhalation during administration. Such administration is typically through contact by the composition comprising the intranasal bacterial polysaccharide conjugate nanoemulsion vaccine with the nasal mucosa, nasal turbinates or sinus cavity. Administration by inhalation comprises intranasal administration, or may include oral inhalation. Such administration may also include contact with the oral mucosa, bronchial mucosa, and other epithelia.

Exemplary dosage forms for pharmaceutical administration are described herein. Examples include but are not limited to liquids, ointments, creams, emulsions, lotions, gels, bioadhesive gels, sprays, aerosols, pastes, foams, sunscreens, capsules, microcapsules, suspensions, pessary, powder, semi-solid dosage form, etc.

The intranasal bacterial polysaccharide conjugate nanoemulsion vaccines may be formulated for immediate release, sustained release, controlled release, delayed release, or any combinations thereof, into the epidermis or dermis. In some embodiments, the formulations may comprise a penetration-enhancing agent. Suitable penetration-enhancing agents include, but are not limited to, alcohols such as ethanol, triglycerides and aloe compositions. The amount of the penetration-enhancing agent may comprise from about 0.5% to about 40% by weight of the formulation.

The pharmaceutical intranasal bacterial polysaccharide conjugate nanoemulsion vaccines for administration may be applied in a single administration or in multiple administrations.

An exemplary nanoemulsion vaccine adjuvant according to the invention is designated “W₈₀5EC” adjuvant. The composition of W₈₀5EC adjuvant is shown in the table below (Table 1). The mean droplet size for the W₈₀5EC adjuvant is ˜400nm. All of the components of the nanoemulsion are included on the FDA inactive ingredient list for Approved Drug Products.

TABLE 1
W805EC Formulation

| | W805EC-Adjuvant |
|---|---|
| Function | Mean Droplet Size ≈ 400 nm |
| Aqueous Diluent | Purified Water, USP |
| Hydrophobic Oil (Core) | Soybean Oil, USP (super refined) |
| Organic Solvent | Dehydrated Alcohol, USP (anhydrous |
| | ethanol) |
| Surfactant | Polysorbate 80, NF |
| Emulsifying Agent Preservative | Cetylpyridinium Chloride, USP |

The nanoemulsion vaccine adjuvants are formed by emulsification of an oil, purified water, nonionic detergent, organic solvent and surfactant, such as a cationic surfactant. An exemplary specific nanoemulsion adjuvant is designated as “60% W₈₀5EC”. The 60% W₈₀5EC-adjuvant is composed of the ingredients shown in Table 2 below: purified water, USP; soybean oil USP; Dehydrated Alcohol, USP [anhydrous ethanol]; Polysorbate 80, NF and cetylpyridinium chloride, USP (CPC). All components of this exemplary nanoemulsion are included on the FDA list of approved inactive ingredients for Approved Drug Products.

TABLE 2
Composition of 60% W805EC-Adjuvant (w/w %)

| Ingredients | 60% W805EC |
|---|---|
| Purified Water, USP | 54.10% |
| Soybean Oil, USP | 37.67% |
| Dehydrated Alcohol, USP | 4.04% |
| (anhydrous ethanol) | |
| Polysorbate 80, NF | 3.55% |
| Cetylpyridinium Chloride, USP | 0.64% |

### V. Methods of Manufacture

The nanoemulsion vaccine adjuvants can be formed using classic emulsion forming techniques. See e.g., U.S. 2004/0043041. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5, 103,497 and 4,895,452, herein incorporated by reference in their entireties.

In an exemplary embodiment, the nanoemulsions used in the methods of the invention comprise droplets of an oily discontinuous phase dispersed in an aqueous continuous phase, such as water or PBS. The nanoemulsions of the invention are stable, and do not deteriorate even after long storage periods. Certain nanoemulsions of the invention are non-toxic and safe when swallowed, inhaled, or contacted to the skin of a subject.

The compositions of the invention can be produced in large quantities and are stable for many months at a broad range of temperatures. The nanoemulsion can have textures ranging from that of a semi-solid cream to that of a thin lotion, to that of a liquid and can be applied topically by any pharmaceutically acceptable method as stated above, e.g., by hand, or nasal drops/spray.

As stated above, at least a portion of the emulsion may be in the form of lipid structures including, but not limited to, unilamellar, multilamellar, and paucliamellar lipid vesicles, micelles, and lamellar phases.

The present invention contemplates that many variations of the described nanoemulsions will be useful in the methods of the present invention. To determine if a candidate nanoemulsion is suitable for use with the present invention, three criteria are analyzed. Using the methods and standards described herein, candidate emulsions can be easily tested to determine if they are suitable. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use. The nanoemulsion of the invention can be provided in many different types of containers and delivery systems. For example, in some embodiments of the invention, the nanoemulsions are provided in a cream or other solid or semi-solid form. The nanoemulsions of the invention may be incorporated into hydrogel formulations.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles and any suitable pressurized spray device. Such spray bottles may be suitable for delivering the nanoemulsions intranasally or via inhalation.

These nanoemulsion-containing containers can further be packaged with instructions for use to form kits.

### VI. Definitions

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the term “adjuvant” refers to an agent that increases the immune response to an antigen (e.g., a pathogen). As used herein, the term “immune response” refers to a subject's (e.g., a human or another animal) response by the immune system to immunogens (i.e., antigens) which the subject's immune system recognizes as foreign. Immune responses include both cell-mediated immune responses (responses mediated by antigen-specific T cells and non-specific cells of the immune system) and humoral immune responses (responses mediated by antibodies present in the plasma lymph, and tissue fluids). The term “immune response” encompasses both the initial responses to an immunogen (e.g., a pathogen) as well as memory responses that are a result of “acquired immunity.”

The terms “chelator” or “chelating agent” refer to any materials having more than one atom with a lone pair of electrons that are available to bond to a metal ion.

As used herein, the term “enhanced immunity” refers to an increase in the level of acquired immunity to a given pathogen following administration of a vaccine of the present invention relative to the level of acquired immunity when a vaccine of the present invention has not been administered.

As used herein, the term “immunogen” refers to an antigen that is capable of eliciting an immune response in a subject. In preferred embodiments, immunogens elicit immunity against the immunogen (e.g., a pathogen or a pathogen product) when administered in combination with a nanoemulsion of the present invention.

As used herein, the term “intranasal(ly)” refers to application of the compositions of the present invention to the surface of the skin and mucosal cells and tissues of the nasal passages, e.g., nasal mucosa, sinus cavity, nasal turbinates, or other tissues and cells which line the nasal passages.

The term “nanoemulsion,” as used herein, includes small oil-in-water dispersions or droplets, as well as other lipid structures which can form as a result of hydrophobic forces which drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The present invention contemplates that one skilled in the art will appreciate this distinction when necessary for understanding the specific embodiments herein disclosed.

The terms “pharmaceutically acceptable” or “pharmacologically acceptable,” as used herein, refer to compositions that do not substantially produce adverse allergic or adverse immunological reactions when administered to a host (e.g., an animal or a human). Such formulations include any pharmaceutically acceptable dosage form. Examples of such pharmaceutically acceptable dosage forms include, but are not limited to, dips, sprays, seed dressings, stem injections, lyophilized dosage forms, sprays, and mists. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

The invention is further described by reference to the following examples, which are provided for illustration only. The invention is not limited to the examples, but rather includes all variations that are evident from the teachings provided herein. All publicly available documents referenced herein, including but not limited to U.S. patents, are specifically incorporated by reference.

### EXAMPLES

### Example 1. Efficacy of Intranasal Polysaccharide Conjugated Nanoemulsion Vaccine in an Animal Model

The goal of this study was to ascertain the efficacy of an intranasal polysaccharide conjugate nanoemulsion vaccine to protect against subsequent infection by Streptococcus pneumoniae in a mouse model.

### 1.1 Vaccine Structure and Formulations

Pn18c was conjugated to CRM197. Protein conjugation may be performed using any conventional protein conjugation technique. Methods of protein conjugation are well-known in the art. Exemplary methods of protein conjugation include, but are not limited to, those described in Sarkar & Jayaraman, “Glycoconjugations of Biomolecules by Chemical Methods,” Front. Chem., 2020 (doi.org/10.3389/fchem.2020.570185); Berti & Adamo, “Antimicrobial glycoconjugate vaccines: an overview of classic and modern approaches for protein modification,” Chem. Soc. Rev., 47:9015 (2018); Ada & Isaacs, “Carbohydrate-protein conjugate vaccines,” Clin. Microbiol. Infect., 9(2):79-85 (2003); Lees et al., “Versatile and efficient synthesis of protein-polysaccharide conjugate vaccines using aminooxy reagents and oxime chemistry,” Vaccine, 24(6):716-29 (2006), each of which is incorporated by reference in its entirety.

The nanoemulsion was formulated by high-speed emulsification of ultra-pure soybean oil with cetylpyridinium chloride, Tween 80, and ethanol in water using a high speed homogenizer. The nanoemulsion contained one part cetylpyridinium chloride and six parts Tween 80.

The vaccine was prepared by mixing of conjugated Pn18C-CRM197 containing 4.0 μg of total polysaccharide with 60% W805EC (NE01) for a final concentration of 20% W805EC.

### 1.2 Treatment (Dosing) and Sample Collection

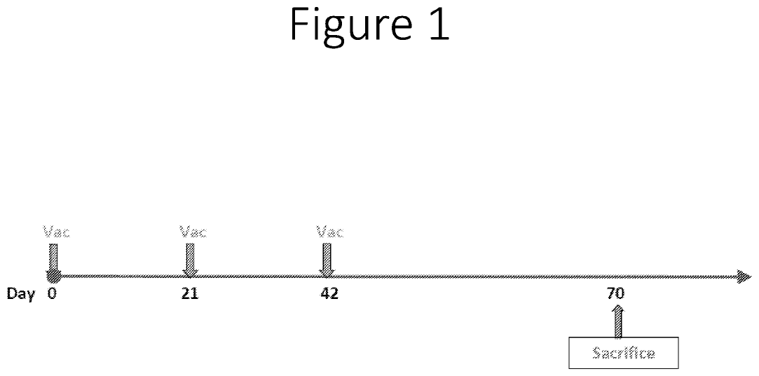

The vaccine formulations were stored at 4° C. prior to use. Mouse immunizations were performed at IBT Bioservices (Rockville, MD) under approved IACUC animal study protocol. Two groups of 7 female CD-1 mice (six to eight weeks old) were either intranasally (IN) immunized with 12 μL of NE01/Pn18c-CRM197 at day 0, 21, 42 or were administered PBS IN (control). The treatment schedule was as given in FIG. 1.

Mice were sacrificed on day 70 post-first dose. Following sacrifice, tissue samples were collected for subsequent analysis. Samples included spleen and lung samples (isolated and homogenized to single cell suspension), serum, and broncho alveolar lavage (BAL) fluid.

### 1.3 Serum and BAL IgG/IgA Titers

Immune responses in mice were measured by determining the IgG and IgA antibody titers against Pn18c and CRM197 in serum and BAL fluid samples from immunized mice following administration of the polysaccharide conjugate nanoemulsion vaccine. IgG and IgA titers were determined using enzyme-linked immunosorbent (ELISA) assays. Briefly, 96-well Immulon 4HBX plates were coated with either 1 μg/mL of Pn18C or CRM197, blocked with 5% BSA/PBS, and serum or BAL samples were added to plates followed by two-fold serial dilutions in 2% BSA/PBS. Antibodies were detected with either Anti-Mouse IgG-HRP (Jackson Immunoresearch 515-035-071) or Rabbit Anti-Mouse IgA-HRP (Rockland 610-4306). The endpoint titer (EPT) was determined by extrapolating the OD values from the dilution points spanning the cutoff value (3 times the mean of background) and calculating the average. OD₄₅₀ measurements were obtained at a sample dilution of 1:5 in PBS.

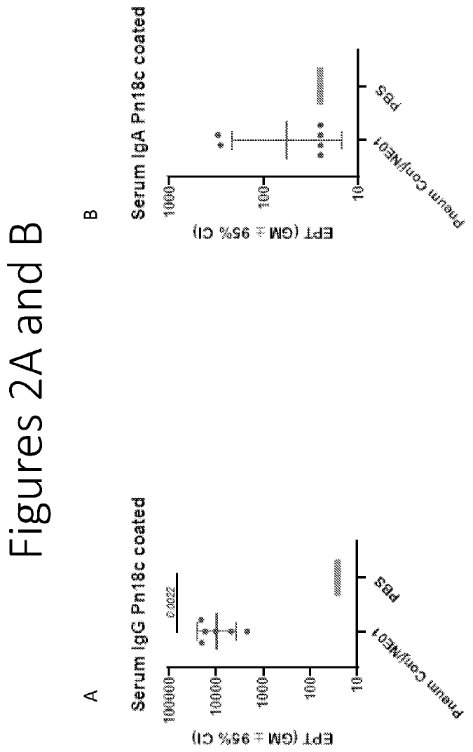

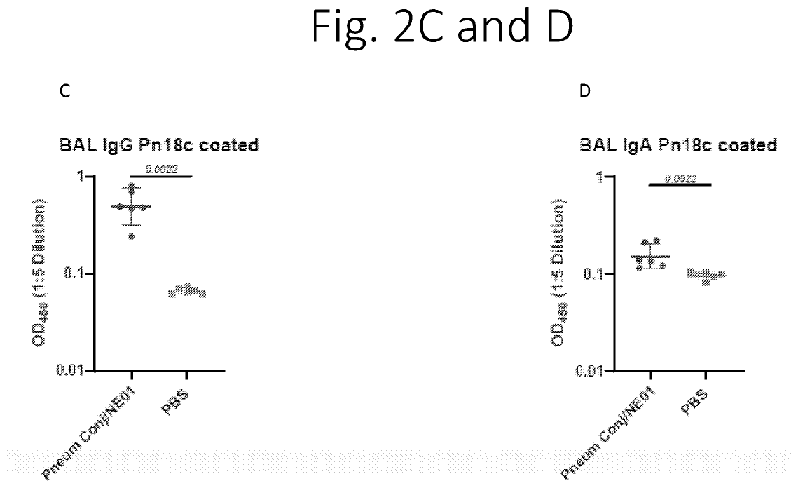

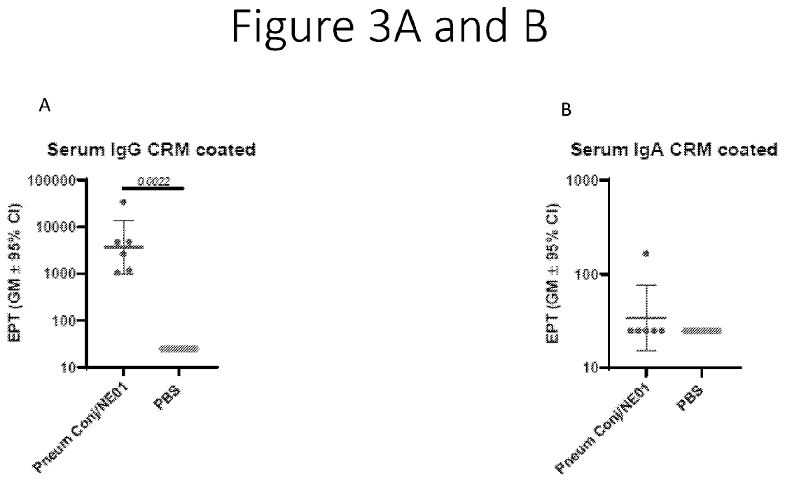

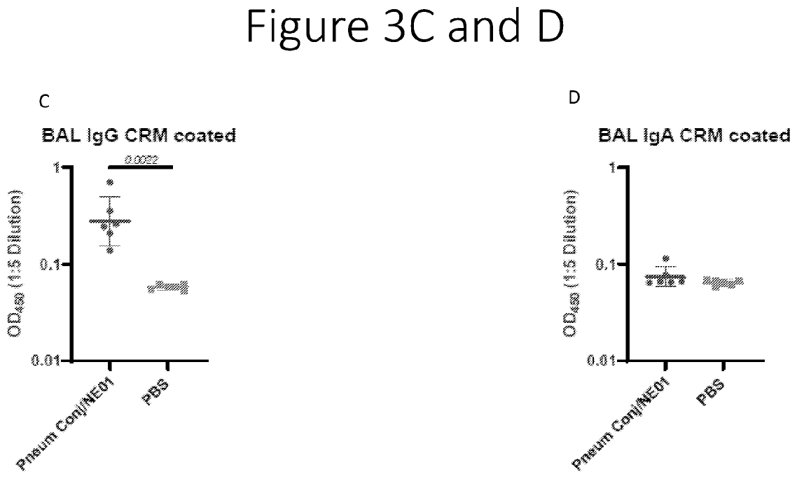

Pneum Conj/NE01 induced robust Pn18c-directed IgG but not IgA antibody titers, shown in FIG. 2A and FIG. 2B, respectively, in serum samples obtained from immunized mice. In BAL samples, significant induction of Pn18c-directed IgG and IgA antibody titers was observed (FIG. 2C and FIG. 2D). Serum and BAL samples were also assayed for CRM-directed antibody titers. As observed for Pn18c, Pneum Conj/NE01 NE01 induced robust CRM-directed IgG but not IgA antibody titers, shown in FIG. 3A and FIG. 3B, respectively. A similar pattern of CRM antibody titer induction was observed in BAL samples (FIG. 3C and FIG. 3D). These experiments indicated that the Pneum Conj/NE01 is capable of inducing a protective immune response against Pn18c.

### 1.4 Lung and Spleen Cytokine Levels

Individual single cell suspensions obtained from dissected mice lungs and spleens were assayed for cytokine levels. Cell suspensions were processed by removal of contaminating red blood cells by using 0.8% ammonium chloride with EDTA, washed with media, and resuspended and plated on a 96 well plate at 5×105 cells per well. Cells were stimulated with CRM197 or Pn18c for 72 hours. Supernatants were collected and Luminex assays were performed according to the manufacturer's protocol.

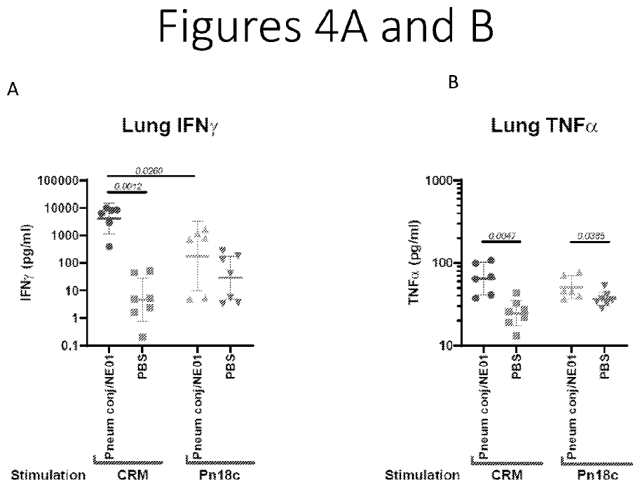

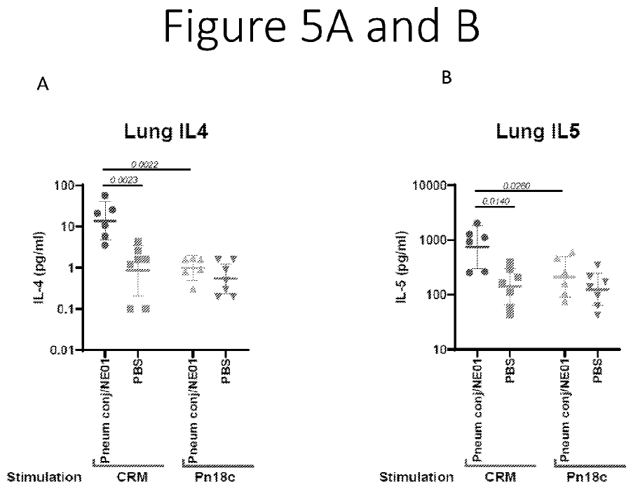

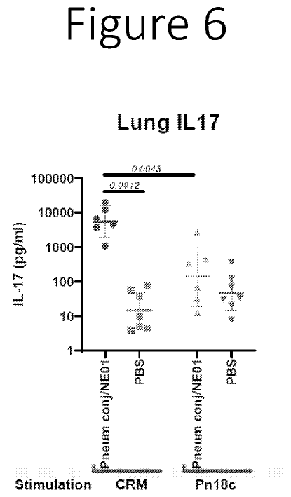

Lung-derived cells from mice immunized with Pneum conj/NE01 and stimulated with CRM exhibited enhanced production of Th1 (IFNγ and TNFα) (FIG. 4A and FIG. 4B), Th2 (IL4 and IL5) (FIG. 5A and FIG. 5B), and Th17 (IL17) (FIG. 6) cytokines relative to levels observed in cells derived from tissue from control mice. Lung-derived cells stimulated with Pn18c did not exhibit enhanced production of Th1, Th2, or Th7 cytokines relative to levels observed in cells derived from lung tissue from control mice, with the exception of TNFα (FIG. 4B).

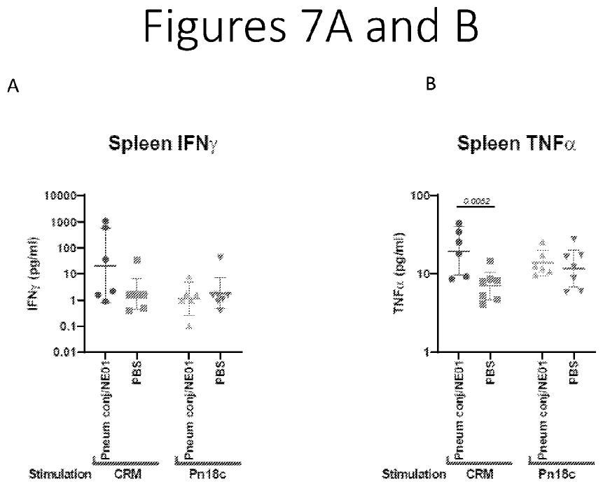

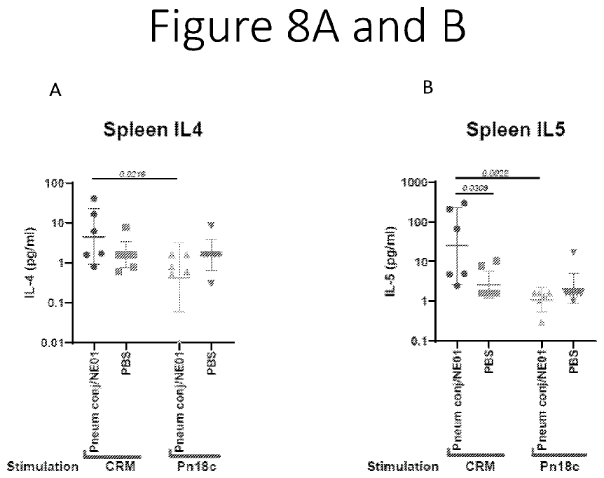

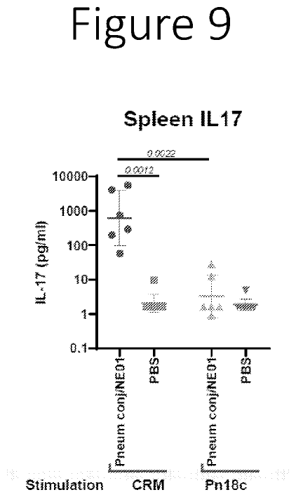

Spleen-derived cells from mice immunized with Pneum conj/NE01 and stimulated with CRM exhibited a modest increase production of Th1 (IFNγ and TNFα) (FIG. 7A and FIG. 7B) and Th2 (IL4 and IL5) (FIG. 8A and FIG. 8B), and a robust enhancement of the production of Th17 (IL17) (FIG. 9) cytokines relative to levels observed in cells derived from spleen tissue from control mice. Spleen-derived cells stimulated with Pn18c did not exhibit enhanced production of Th1, Th2, or Th7 cytokines relative to levels observed in cells derived from tissue from control mice.

### 1.5 Homing of B-Cells to Lungs and Spleens

In order to measure homing of IgG- and IgA-producing B cells to the lungs and spleens of immunized mice, ELISpot assays were performed. Briefly, single cell suspensions of lung and spleen tissue harvested from mice, as described above, were stimulated with IL-2 (0.5 μg/mL) and RD848 (1 μg/mL) for 3 days followed by washing and plating onto PVDF ELISpot filter plates coated with anti-mouse IgG or IgA. The cells were incubated for 24 hours at 37° C., followed by the addition of biotinylated Pn18C. Anti-IgG or Anti-IgA antigen specific cells were detected using streptavidin-HRP. The spots were quantified via AID ELISpot reader.

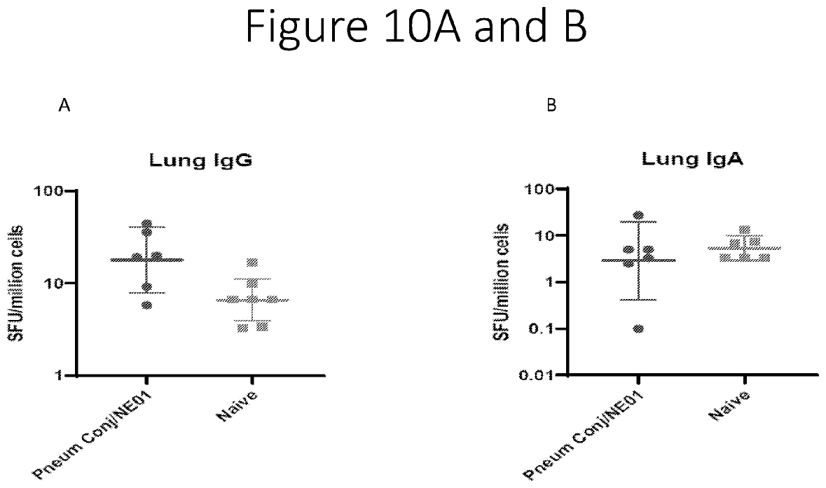

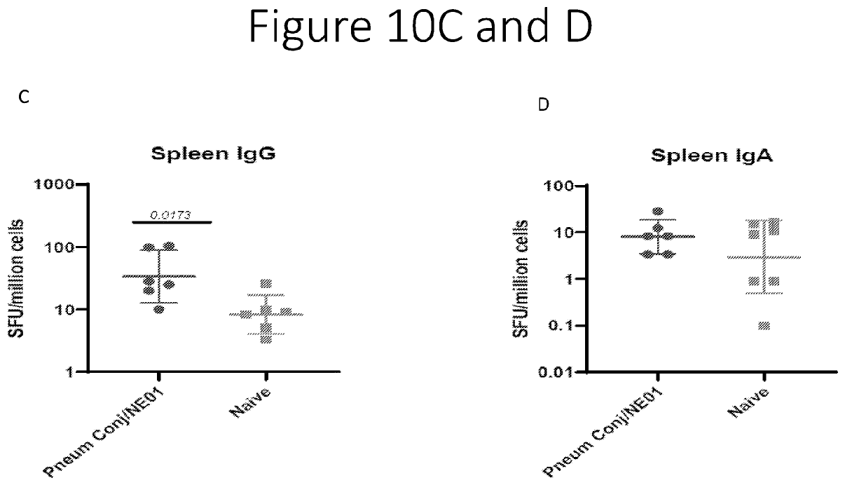

Pneum Conj/NE01 promoted the homing of IgG-producing B cells to the lungs (FIG. 10A) and spleens (FIG. 10C) of immunized mice, as compared to naïve mice. No significant homing of IgA-producing B cells to the lungs (FIG. 10B) and spleens (FIG. 10D) of immunized mice was observed.

### 1.6 Opsonophagocytosis Assay

The OPKA was performed as previously described (Burton & Nahm, Clin Vaccine Immunol, 2006; 13:1004-9). Briefly, 4.5×10⁵ HL-60 cells/mL were differentiated for 5 days using 0.8% DMF in 10% FBS RPMI-1640 media and adjusted to a final concentration of 1.0×10⁷ cells/mL after washing. Heat-inactivated sera from immunized or control mice was serially 3-fold diluted on a 96 well plate and incubated with 10 uL of 5×10⁴ CFU of S. pneumoniae strain OREP18 and incubated for 30 min at room temperature. 10 μL of baby rabbit complement and 40 μL of differentiated HL-60 cells were incubated for 45 min at 37° C. 5% CO2. The assay was stopped by placing the plate on ice for 20 min. 5 μL from each well was spotted on a Todd-Hewitt Broth, Yeast (THBY) agar plate followed by an THBY overlay containing triphenyl tetrazolium chloride (TTC). The plates were incubated overnight at 37° C. 5% CO² incubator. The colonies were counted and opsonization titers were determined from the interpolation of the serum dilution spanning 50% killing cutoff determined from S. pneumoniae subjected to assay conditions without heat inactivated serum.

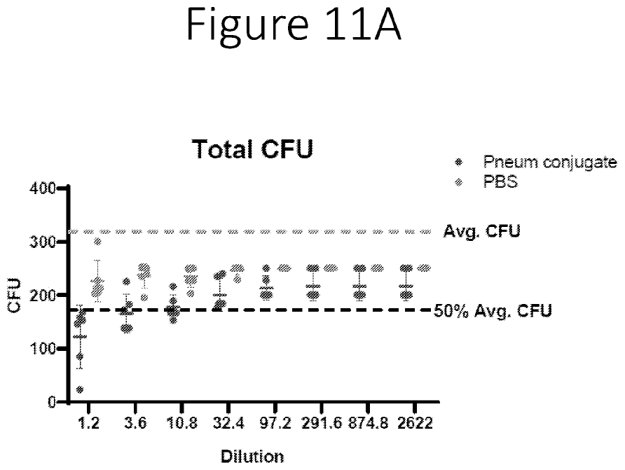

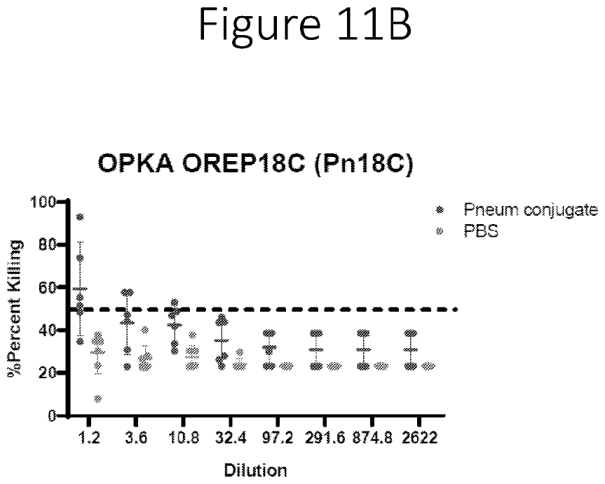

Sera isolated from mice immunized with Pneum-Conj/NE01 exhibited a reduction in total colony forming units (CFU) over the entire range of tested dilutions, relative to sera from control mice (FIG. 11A). This result was indicative of an increase in the Percent Killing of S. pneumonia by sera from immunized mice (FIG. 11B).

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. An intranasal bacterial vaccine composition comprising:
(a) a polysaccharide-antigen conjugate comprising:
(i) one or more polysaccharides from at least one polysaccharide-encapsulated bacteria, and
(ii) a carrier protein covalently linked to one or more polysaccharides; and
(b) a nanoemulsion adjuvant comprising:
(i) droplets having an average diameter of less than about 1000 nm;
(ii) an aqueous phase;
(iii) about 1% to about 80% of at least one pharmaceutically acceptable oil;
(iv) about 0.001% to about 10% of at least one surfactant, wherein the surfactant is a polyoxyethylene nonionic surfactant, a cationic quaternary ammonium compound, or a combination thereof; and
(v) about 0.1% to about 50% of at least one organic solvent, wherein the organic solvent is an alcohol.

2. The intranasal bacterial vaccine composition of claim 2, wherein the composition comprises a panel of at least two polysaccharides from two different bacterial serotypes from the same bacterial genus and species.

3. The intranasal bacterial vaccine composition of claim 1, wherein the bacteria is selected from the group consisting of Haemophilus, Neissera, Streptococcus, Shigella, Salmonella, and Porphyromonas.

4. The intranasal bacterial vaccine composition of claim 3, wherein the bacteria is selected from the group consisting of Haemophilus influenzae B (Hib), Neissera meningitides, Streptococcus pneumonia (pneumococcus), Shigella dysenteriae, Shigella flexneri, Shigella boydii, Shigella sonnei, Salmonella typhi, Salmonella paratyphi, and Salmonella enterica, and Porphyromonas gingivalis.

5. The intranasal bacterial vaccine composition of claim 3, wherein the at least one polysaccharide-encapsulated bacteria is from the group consisting of:
(a) one or more Streptococcus pneumonia serotypes;
(b) a combination of serotypes 1, 2, 3, 4, 5, 6B, 7F, 8, 9N, 9V, 10A, 11A, 12F, 14, 15B, 17F, 18C, 19F, 19A, 20, 22F, 23F, and 33F of Streptococcus pneumonia,
(c) a combination of serotypes 1, 3, 4, 5, 6A, 6B, 7F, 9V, 14, 18C, 19A, 19F, and 23F Streptococcus pneumonia;
(d) one or more of serotypes 19A, 6, 3, 23F of Streptococcus pneumonia;
(e) one or more of serotypes 4, 6B, 9V, 14, 18C, 19F, and 23F of Streptococcus pneumonia;
(f) one or more of serotypes 6A, 19A, and 15B/C of Streptococcus pneumonia; and
(g) one or more of serotypes 1, 2, 3, 4, 5, 6A, 6B, 6C, 7C, 7F, 8, 9A, 9N, 9A/N, 9N/L, 9V, 10A, 11A, 12F, 14, 15A, 15B, 15A/B, 15C, 16F, 17F, 18B, 18C, 19A, 19F, 19A, 20, 21A, 22F, 23A, 23B, 23 A/B, 23F, 24F, 31, 33F, 35A/B/C, 35B, 38 of Streptococcus pneumonia.

6. The intranasal bacterial vaccine composition of claim 3, wherein the at least one polysaccharide-encapsulated bacteria is from the group consisting of:
(a) one or more serogroups of Neisseria meningitides;
(b) one or more of serogroups A, B, C, W, W-135, X, and Y of Neisseria meningitides; and
(c) one or more of serogroups A, B, C, D, H, I, K, N, W, W-135, X, and Y of Neisseria meningitides.

7. The intranasal bacterial vaccine composition of claim 3, wherein the at least one polysaccharide-encapsulated bacteria is from the group consisting of:
(a) one or more serogroups of Shigella dysenteriae,
(b) one or more serogroups of Shigella flexneri;
(c) one or more serogroups of Shigella boydii; and/or
(d) one or more serogroups of Shigella sonnei.

8. The intranasal bacterial vaccine composition of claim 3, wherein the at least one polysaccharide-encapsulated bacteria is from the group consisting of:
(a) one or more serotypes of Salmonella enterica;
(b) Salmonella enterica serotype Enteritidis;
(c) Salmonella enterica serotype Typhimurium;
(d) one or more serotypes of Salmonella typhi;
(e) one or more serotypes of Salmonella typhi; and/or
(f) one or more serotypes of Salmonella paratyphi:

9. The intranasal bacterial vaccine composition of claim 3, wherein the at least one polysaccharide-encapsulated bacteria is from one or more serotypes of Porphyromonas gingivalis.

10. The intranasal bacterial vaccine composition of claim 1, wherein the carrier protein is selected from the group consisting of diphtheria toxoid (DT), CRM197 diphtheria toxin, tetanus toxoid (TT), CRM197, Haemophilus protein D (PD), and the outer membrane protein complex of serogroup B meningococcus (OMPC).

11. The intranasal bacterial vaccine composition of claim 1, wherein the polysaccharide-conjugated antigen comprises bacterial capsular polysaccharides (CPSs).

12. The intranasal bacterial vaccine composition of claim 1, wherein the amount of the polysaccharide per dose and per bacterial strain, is greater than about 1 μg and less than about 40 μg.

13. The intranasal bacterial vaccine composition of claim 1, wherein the nanoemulsion adjuvant and/or the vaccine composition:
(a) is not systemically toxic to the subject; and/or
(b) produces minimal or no inflammation upon administration.

14. The intranasal bacterial vaccine composition of claim 1, wherein the nanoemulsion adjuvant comprises droplets having an average diameter selected from the group consisting of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, greater than about 125 nm and less than about 600 nm, and any combination thereof.

15. The intranasal bacterial vaccine composition of claim 14, wherein:
(a) the nanoemulsion adjuvant comprises droplets having an average diameter of less than about 800 nm; or
(b) the nanoemulsion adjuvant comprises droplets having an average diameter of less than about 600 nm; or
(c) the nanoemulsion adjuvant comprises droplets having an average diameter of less than about 500 nm; or
(d) the nanoemulsion adjuvant comprises droplets having an average diameter of less than about 400 nm.

16. The intranasal bacterial vaccine composition of claim 1, wherein administration of the vaccine composition to a subject results in seroconversion of the subject after a single administration of the vaccine composition.

17. The intranasal bacterial vaccine composition of claim 1, wherein the alcohol is:
(a) selected from the group consisting of a C₁-C₁₂ alcohol, diol, triol, and combinations thereof;
(b) selected from the group consisting of a nonpolar solvent, a polar solvent, a protic solvent, an aprotic solvent, semi-synthetic derivatives thereof, and combinations thereof;
(c) selected from the group consisting of ethanol, methanol, isopropyl alcohol, glycerol, n-butanol, butylene glycol, perfumers alcohols, isopropanol, n-propanol, propylene glycols, glycerol, sorbitol, and any combination thereof; or
(d) any combination of (a), (b), and/or (c).

18. The intranasal bacterial vaccine composition of claim 1, wherein the oil:
(a) is selected from the group consisting of animal oil, vegetable oil, natural oil, synthetic oil, hydrocarbon oils, silicone oils, and semi-synthetic derivatives there; or
(b) is selected from the group consisting of soybean, avocado, squalene, olive, canola, corn, rapeseed, safflower, sunflower, fish, flavor, and water insoluble vitamins.

19. The intranasal bacterial vaccine composition of claim 1, wherein the nonionic polyoxyethylene surfactant is selected from the group consisting of polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, semi-synthetic derivatives thereof, and mixtures thereof.

20. The intranasal bacterial vaccine composition of claim 1, wherein the cationic quaternary ammonium compound is selected from the group consisting of an alkyl trimethyl ammonium chloride compound, a dialkyl dimethyl ammonium chloride compound, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Cetylpyridinium chloride, Dimethyldioctadecylammonium bromide, Dodecylethyldimethylammonium bromide, Dodecyltrimethylammonium bromide, Ethylhexadecyldimethylammonium bromide, Girard's reagent T, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C₁₂), Alkyl dimethyl 3,4-dichlorobenzyl ammonium chloride (50% C₁₄, 40% C₁₂, 10% C₁₆), Alkyl dimethyl 3,4-dichorobenzyl ammonium chloride (55% C₁₄, 23% C₁₂, 20% C₁₆), Alkyl dimethyl benzyl ammonium chloride, Alkyl dimethyl benzyl ammonium chloride (100% C₁₄), Alkyl dimethyl benzyl ammonium chloride (100% C₁₆), Alkyl dimethyl benzyl ammonium chloride (41% C₁₄, 28% C₁₂), Alkyl dimethyl benzyl ammonium chloride (47% C₁₂, 18% C₁₄), Alkyl dimethyl benzyl ammonium chloride (55% C₁₆, 20% C₁₄), Alkyl dimethyl benzyl ammonium chloride (58% C₁₄, 28% C₁₆), Alkyl dimethyl benzyl ammonium chloride (60% C₁₄, 25% C₁₂), Alkyl dimethyl benzyl ammonium chloride (61% C₁₁, 23% C₁₄), Alkyl dimethyl benzyl ammonium chloride (61% C₁₂, 23% C₁₄), Alkyl dimethyl benzyl ammonium chloride (65% C₁₂, 25% C₁₄), Alkyl dimethyl benzyl ammonium chloride (67% C.sub.12, 24% C₁₄), Alkyl dimethyl benzyl ammonium chloride (67% C₁₂, 25% C₁₄), Alkyl dimethyl benzyl ammonium chloride (90% C₁₄, 5% C₁₂), Alkyl dimethyl benzyl ammonium chloride (93% C₁₄, 4% C₁₂), Alkyl dimethyl benzyl ammonium chloride (95% C₁₆, 5% C₁₈), Alkyl didecyl dimethyl ammonium chloride, Alkyl dimethyl benzyl ammonium chloride (C₁₂₋₁₆), Alkyl dimethyl benzyl ammonium chloride (C₁₂₋₁₈), dialkyl dimethyl benzyl ammonium chloride, Alkyl dimethyl dimethybenzyl ammonium chloride, Alkyl dimethyl ethyl ammonium bromide (90% C₁₄, 5% C₁₆, 5% C₁₂), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C₁₄), Alkyl dimethyl isopropylbenzyl ammonium chloride (50% C₁₂, 30% C₁₄, 17% C₁₆, 3% C₁₈), Alkyl trimethyl ammonium chloride (58% C₁₈, 40% C₁₆, 1% C₁₄, 1% C₁₂), Alkyl trimethyl ammonium chloride (90% C₁₈, 10% C₁₆), Alkyldimethyl(ethylbenzyl) ammonium chloride (C₁₂₋₁₈), Di-(C₈₋₁₀)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof.

21. The intranasal bacterial vaccine composition of claim 1, wherein:
(a) the nanoemulsion adjuvant comprises at least one cationic surfactant and at least one polyoxyethylene nonionic surfactant;
(b) the nanoemulsion adjuvant comprises at least one cationic surfactant and at least one polyoxyethylene nonionic surfactant which is polysorbate 20, polysorbate 80, poloxamer 188, poloxamer 407, or a combination thereof;
(c) the nanoemulsion adjuvant comprises at least one cationic surfactant and at least one polyoxyethylene nonionic surfactant which is polysorbate 20, polysorbate 80, poloxamer 188, poloxamer 407, or a combination thereof, and wherein the polyoxyethylene nonionic surfactant is present at about 0.01% to about 5.0%, or at about 0.1% to about 3%;
(d) the nanoemulsion adjuvant comprises at least one cationic surfactant and at least one polyoxyethylene nonionic surfactant, and the non-ionic surfactant is present in a concentration of about 0.05% to about 10%, about 0.05% to about 7.0%, about 0.1% to about 7%, or about 0.5% to about 4%;
(e) the nanoemulsion adjuvant comprises at least one cationic surfactant and at least one polyoxyethylene nonionic surfactant, wherein the cationic surfactant is present in a concentration of about 0.05% to about 2% or about 0.01% to about 2%; or
(f) any combination of (a)-(f).

22. The intranasal bacterial vaccine composition of claim 1, further comprising chitosan.

23. The intranasal bacterial vaccine composition of claim 1, further comprising glucan.

24. The intranasal bacterial vaccine composition of claim 1, wherein the aqueous phase is present in Phosphate Buffered Saline (PBS).

25. The intranasal bacterial vaccine composition of claim 1, wherein the composition is formulated:
(a) into a dosage form selected from the group consisting of a liquid dispersion, aerosol, and suspensions; and/or
(b) into a controlled release formulation, sustained release formulation, or immediate release formulation.

26. A method for inducing an enhanced immunity against disease caused by a polysaccharide-encapsulated bacteria comprising the step of administering to a subject an effective amount of the intranasal bacterial vaccine composition of claim 1.

27. The method of claim 26, wherein the subject produces a protective immune response after at least a single administration of the vaccine composition.

28. The method of claim 26, wherein the immune response is protective against one or more serotypes of the bacteria.

29. The method of claim 26, wherein the subject exhibits a higher titer of bacteria-specific antibodies relative to a subject not administered the intranasal bacterial vaccine composition.

30. The method of claim 29, wherein the bacteria-specific antibodies comprise IgG antibodies.

31. The method of claim 29, wherein the bacteria-specific antibodies comprise IgA antibodies.

32. The method of claim 26, wherein the subject exposed to the intranasal bacterial vaccine composition exhibits elevated serum levels of IFN-γ as compared to the levels found within a subject not administered the intranasal bacterial vaccine composition.

33. The method of claim 26, wherein the subject exposed to the intranasal bacterial vaccine composition exhibits elevated serum levels of TNF-α as compared to the levels found within a subject not administered the intranasal bacterial vaccine composition.

34. The method of claim 26, wherein the subject exposed to the intranasal bacterial vaccine composition exhibits elevated serum levels of IL-4 as compared to the levels found within a subject not administered the intranasal bacterial vaccine composition.

35. The method of claim 26, wherein the subject exposed to the intranasal bacterial vaccine composition exhibits elevated serum levels of IL-5 as compared to the levels found within a subject not administered the intranasal bacterial vaccine composition.

36. The method of claim 26, wherein the subject exposed to the intranasal bacterial vaccine composition exhibits elevated serum levels of IL-17 as compared to the levels found within a subject not administered the intranasal bacterial vaccine composition.

37. The method of claim 32, wherein the elevated levels are measured in the lung and/or spleen of the subject.
